(19) United States
(12) Patent Application Publication
(10) Pub. No.: US 2026/0144723 A1
(43) Pub. Date: May 28, 2026

(54) HAIR CLEANSER FORMULATION FOR TEXTURED HAIR

(71) Applicant: TheKlayCo. LLC, Cleveland, OH (US)

(72) Inventor: Kaelynn Workman, CLEVELAND, OH (US)

(73) Assignee: TheKlayCo. LLC, Cleveland, OH (US)

(21) Appl. No.: 18/956,394

(22) Filed: Nov. 22, 2024

(51) Int. Cl. A61K 8/25; A61K 8/365; A61Q 5/02

(52) U.S. Cl. CPC A61K 8/25; A61K 8/365; A61Q 5/02

(57) ABSTRACT

A hair cleanser formulation for textured hair includes from 15% to 25% by weight of one or more clays, and from 3% to 7% by weight of glycolic acid. The glycolic acid combines with the clays from 18% to 32% by weight of the of the formulation. Supporting ingredients make up the remaining total volume (% w/w) of the formulation.

### BACKGROUND

Textured hair, that is hair having a curl pattern, has unique hair care needs. In particular, understanding the distinct biological and structural characteristics of afro-textured hair (i.e., African descent hair) is crucial for effectively caring for this delicate hair type. Generally, afro-textured hair is tightly curled or coiled and includes hair types that are classified as 3a-4c on the Andre Walker hair scale. Characteristics of afro-textured hair include, but are not limited to, ribbon-like cross-sections of the hair shaft, elliptically shaped hair follicles, varying diameters throughout the hair shaft, and impaired hydrophobicity with high water intake. This unique structure can make it more prone to damage. For example, afro-textured hair is more prone to proximal trichorrhexis nodosa (i.e., hair breakage near the scalp), formation of complex knots, dryness, and the development of trichodystrophies (i.e., hair shaft damage), among others. Because of its delicate nature, certain processes involved in hair care maintenance can exacerbate damage of afro-textured hair, for example, the washing process.

Generally, washing the hair with shampoo helps to remove dirt and sebum from the scalp and hair. Some shampoos use anionic surfactants as a cleansing detergent. Anionic surfactants are strong and effective cleansers. However, anionic surfactants are very harsh on the hair shaft. Anionic surfactants are characterized by their negatively charged polar hydrophilic groups. During the washing process, the negatively charged ions from the cleansing detergent will deposit on the hair shaft, increasing the negative net electrical charge, which leads to static electricity and increased friction between the hair fibers. This causes the hair fibers to repel from one another resulting in frizz and hair damage. Accordingly, the harsh and drying nature of anionic surfactants are not ideal for afro-textured hair.

Other categories of surfactants include cationic, nonionic and amphoteric surfactants. Cationic surfactants are noted for their minimal cleansing capacity and potential to cause buildup on the hair fiber. Non-ionic surfactants are also known for their low cleansing properties and high conditioning properties. Some hair cleansers use alternative cleansing detergents, other than anionic surfactants, primarily amphoteric surfactants. Amphoteric surfactants display different properties based on the pH of the solution it is in. In acidic solutions, amphoteric surfactants commonly behave like cationic surfactants. In alkaline solutions, amphoteric surfactants behave closer to anionic surfactants. Thus, these cleansers may require increased concentration, combination with other surfactants or an alkaline or basic solution for effective cleansing capacity, depending on their formulation. This can exacerbate hair breakage in afro-textured hair.

Other hair cleansers use alternative cleansing detergents such as clay and organic acids. Clays fall outside of the category of surfactants and are a naturally occurring material usually composed of aluminum silicates. However, hair cleansers that utilize clays typically have an alkaline or basic pH, which can also exacerbate hair breakage in afro-textured hair. As is known, the pH scale runs from 0-14, with 7 being neutral, values lower than 7 being acidic, and values greater than 7 being alkaline or base. The pH of hair is normally acidic. Specifically, the pH of the hair shaft is 3.67 and the scalp has a pH between 4.5-5.5. Some alternative cleansing detergents fail to consider the pH of the hair shaft and the pH of the scalp. A study from 2014 analyzing the pH of 123 shampoo brands concluded that only 38% of popular brand shampoos had a pH less than 5. The pH of cleansing detergents can influence the electrical charge of the hair fiber surface. Having a product that is too basic can damage the hair shaft because during the washing process the negative charge of the hair fiber surface will increase due to the basic pH, which results in more static electricity and friction between the hair. On the other hand, having a product that is too acidic will over time damage the hair and lead to surface degradation and cuticle contraction. Thus, a hair cleanser that will effectively cleanse the hair and scalp of dirt and sebum while also maintaining the structural integrity (i.e., the cuticle and cortex of the hair shaft to prevent breakage and frizz) of afro-textured hair is desirable.

### BRIEF DESCRIPTION

According to one aspect, a hair cleanser formulation for textured hair includes from 15% to 25% by weight of one or more clays, and from 3% to 7% by weight of glycolic acid. The glycolic acid combines with the clays from 18% to 32% by weight of the of the formulation. Supporting ingredients make up the remaining total volume (% w/w) of the formulation.

### BRIEF DESCRIPTION OF THE DRAWINGS

The accompanying drawings, which are incorporated in and constitute a part of the specification, illustrate various systems, methods, devices, and other embodiments of the disclosure. It will be appreciated that the illustrated element boundaries (e.g., boxes, groups of boxes, directional lines, or other shapes) in the figures represent one embodiment of the boundaries. In some embodiments one element may be designed as multiple elements or that multiple elements may be designed as one element. In some embodiments, an element shown as an internal component of another element may be implemented as an external component and vice versa. Furthermore, elements may not be drawn to scale.

FIG. 1A is a trichoscopy image of hair and scalp fourteen (14) days after a hair wash and immediately before usage of an exemplary hair cleanser formulation according to one embodiment;

FIG. 1B is a trichoscopy image of the hair and scalp shown in FIG. 1A, but after usage of the exemplary hair cleanser formulation according to one embodiment;

FIG. 2 is a box plot showing hair breakage in afro-textured hair when using a commercial shampoo with anionic surfactants verses the exemplary hair cleanser formulation;

FIG. 3 illustrates a flowchart for the method of mixing the hair cleansing formulation; and

FIG. 4 is a chart of an exemplary list of ingredients and formulation percentages according to one embodiment.

### DETAILED DESCRIPTION

The present invention generally relates to a hair cleansing formulation comprising combination of clays, an acid, and a method for mixing the hair cleansing formulation. It is understood that all percentages herein are based upon the total weight of the compositions of the present invention, unless otherwise indicated.

To effectively cleanse the scalp of dirt and sebum, while simultaneously maintaining the structural integrity of the hair shaft (i.e., the cuticle and cortex) to prevent breakage and frizz of afro-textured hair, provided herein is a hair cleansing formulation that generally comprises clays and glycolic acid and has a pH from 3.5 to 3.8, and in some cases, a pH of 3.8±0.01. The clay expands between layers of the hair to remove dirt and excess oil. The glycolic acid loosens dead skin cells that are then removed while washing the hair. The glycolic acid increases the elastic modulus and lubricity of the hair by penetration of the hair shaft, thus strengthening the hair fibers and strands and thereby maintaining the structural integrity of the hair shaft. The lowered pH of the cleansing formulation closely matches that of the hair shaft allowing the hair to have a net neutrality of charges, which reduces hair tangling, unruliness, and frizz during the washing process.

More specifically, the hair cleanser formulation includes from 15 wt. % to 25 wt. % of one or more clays. In one embodiment, the total amount of the one or more clays is 21±0.01 wt. %. In some cases, the total amount of the one or more clays is from 15 wt. % to 24 wt. %; 15 wt. % to 23 wt. %; 15 wt. % to 22 wt. %; 15 wt. % to 21 wt. %; 16 wt. % to 25 wt. %; 16 wt. % to 24 wt. %; 16 wt. % to 23 wt. %; 16 wt. % to 22 wt. %; 16 wt. % to 21 wt. %; 17 wt. % to 25 wt. %; 17 wt. % to 24 wt. %; 17 wt. % to 23 wt. %; 17 wt. % to 22 wt. %; 17 wt. % to 21 wt. %; 18wt. % to 25 wt. %; 18 wt. % to 24 wt. %; 18 wt. % to 23 wt. %; 18 wt. % to 22 wt. %; 18 wt. % to 21 wt. %; 19 wt. % to 25 wt. %; 19 wt. % to 24 wt. %; 19 wt. % to 23 wt. %; 19 wt. % to 22 wt. %; 19 wt. % to 21 wt. %; 20 wt. % to 25 wt. %; 20 wt. % to 24 wt. %; 20 wt. % to 23 wt. %; 20 wt. % to 22 wt. %; 20 wt. % to 21 wt. %.

In one embodiment, the clays include a first clay and a second clay of different types. In one embodiment, the first clay is selected from a smectite clay group and a second clay is selected from a montmorillonite clay group. For example, the first clay is kaolin clay and the second clay is Moroccan clay (i.e., Rhassoul Clay). In one embodiment, the hair cleanser formulation includes 1±0.01 wt. % of kaolin clay, and 20±0.01 wt. % of Moroccan clay.

Additionally, the hair cleanser formulation includes from 3 wt. % to 7 wt. % of glycolic acid. In one embodiment, the total amount of glycolic acid is 3±0.01 wt. %. In some cases, the total amount of glycolic acid is from 3 wt. % to 6 wt. %; 3 wt. % to 5 wt. %; 3 wt. % to 4 wt. %; 4 wt. % to 6 wt. %; 4 wt. % to 5 wt. %.

Together, in one embodiment, the clays and the glycolic acid are 18% to 32% by weight of the formulation. The supporting ingredients, discussed in further detail herein, make up the remaining total volume (% w/w) of the formulation. In some cases, the clays and the glycolic acid are 18 wt. % to 31 wt. %; 18 wt. % to 30 wt. %; 18 wt. % to 29 wt. %; 18 wt. % to 28 wt. %; 18 wt. % to 27 wt. %; 18 wt. % to 26 wt. %; 18 wt. % to 29 wt. %; 18 wt. % to 28 wt. %; 18 wt. % to 27 wt. %; 18 wt. % to 26 wt. %; 18 wt. % to 25 wt. %; 18 wt. % to 24 wt. %; 18 wt. % to 23 wt. %; 18 wt. % to 22 wt. %.

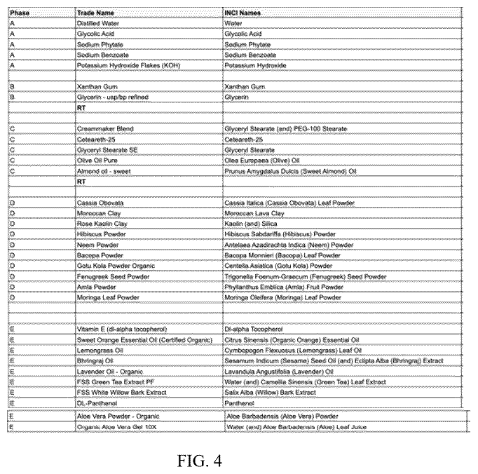

The supporting ingredients can include, but are not limited to, sodium phytate, sodium benzoate, potassium hydroxide, xanthan gum, glyceryl stearate, PEG-100 stearate, olea Europaea (olive) oil, Prunus amygdalus dulus (Sweet Almond) oil, cassia italica leaf powder, hibiscus sabdariffa, antelea azadriratcha indica (neem) powder, and panthenol. Additional supporting ingredients can be seen in FIG. 4.

In one embodiment, the hair cleanser formulation has a pH from 3.5 to 3.8. For example, in one embodiment, the hair cleanser formulation has a pH of 3.8. As discussed above, the pH of the hair shaft is 3.67 and the scalp has a pH between 4.5-5.5. The hair cleanser formulation pH maintains a neutral balance close to the pH of the hair shaft, which aids in reducing friction of the hair shaft and therefore maintain the structural integrity of the hair shaft while still being able properly cleanse the scalp.

The texture of the hair cleanser formulation may be an opaque thick liquid. The liquid-like consistency aids in daily washing and cleansing. This contrasts with highly viscous hair masks, which are thick and sticky, and used less frequently as restorative treatments (e.g., two to three times a week). Typically, these highly viscous hair masks comprise as much as 40% by weight of clays, to aid in the stability and preferential heavy consistency (i.e., high viscosity). As discussed above, preferably, in the hair cleanser formulation, the clays range from 18 wt. % to 32 wt. %. The presence of clay in the hair cleanser formulation reduces pH drift (i.e., slow movement) from the actual, expected pH discussed herein. Higher percentages of clays, as in the highly viscous hair masks, do not reduce pH drift.

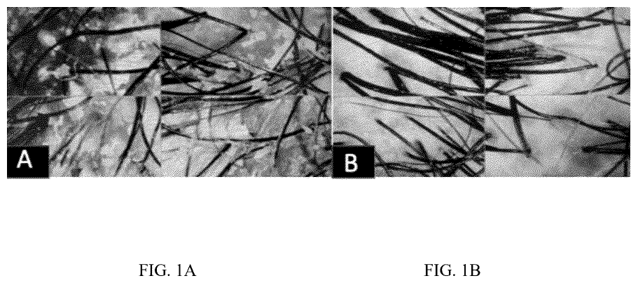

As mentioned above, in one embodiment, the hair cleansing formulation comprises 20±0.01 wt. % Moroccan clay, 1±0.01 wt % kaolin clay, 3±0.01 wt. % glycolic acid; and 76 ±0.01 wt. % supporting ingredients. In this embodiment, the hair cleanser formulation has a pH of 3.8±0.01 An experimental study was performed using this embodiment of the hair cleansing formulation, the results of which will now be described with reference to FIGS. 1A and 1B. FIG. 1A shows four (4) trichoscopy images (50× magnification) of hair and scalp fourteen (14) days after a hair wash and immediately before usage of the hair cleanser formulation according to one embodiment. It is noted that the average time between washes for afro-textured hair is 7-14 days. FIG. 1B shows four (4) trichoscopy images (50× magnification) of the hair and scalp imaged in FIG. 1A after usage of the hair cleanser formulation. As can be seen in FIG. 1B, the hair cleanser formulation efficiently cleared the hair and scalp of oil and debris in comparison to FIG. 1A.

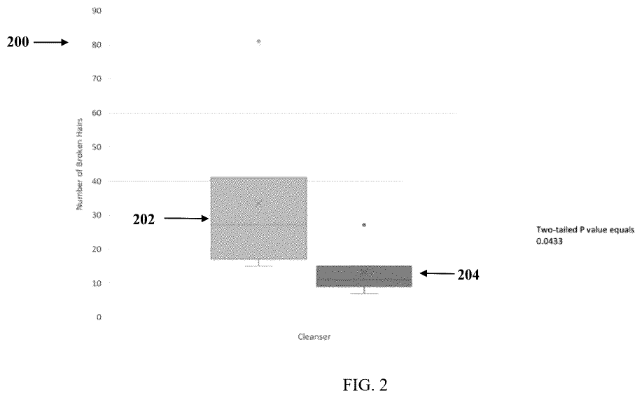

Additionally, rates for hair breakage were studied using the hair cleanser formulation. Two hair tresses of type 3C/4A human virgin hair were randomly assigned treatment with the hair cleanser formulation and treatment with an over-the-counter anionic surfactant based shampoo. The hair tresses underwent seven (7) rounds of hair washing and comb testing. Amounts of hair breakage were collected and counted after every wash. The results of which were analyzed using a two-tailed T test which is shown in FIG. 2. More specifically, FIG. 2 illustrates a box plot chart 200 where box 202 represents the over-the-counter anionic surfactant based shampoo and box 204 represents the hair cleanser formulation. As can be seen, hair breakage was significantly decreased in the hair tress washed with the hair cleanser formulation compared to the over-the-counter anionic surfactant based shampoo.

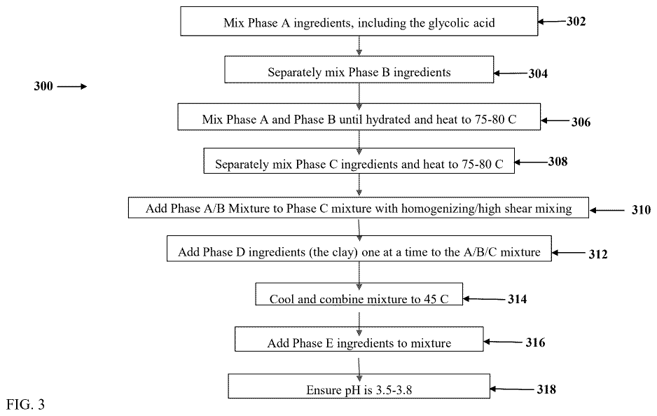

Also provided herein is the method of mixing the hair cleansing formulation. The mixture consists of five (5) phases, each combining different ingredients. The ingredients of each phase can be seen in FIG. 4. Referring now to FIG. 3 and with reference to the phases in FIG. 4, a method 300 for mixing the hair cleansing formulation is shown.

At block 302, the method 300 includes mixing Phase A components, namely, distilled water, glycolic acid, Sodium Phytate, sodium Benzoate, and Potassium Hydroxide. At block 304, the method 300 includes separately mixing Phase B components, namely, Xanthan Gum and Glycerin. At block 306, the method 300 includes mixing Phase A and Phase B ingredients until hydrated and heating to 75-80 degrees Celsius. At block 308, the method 300 includes separately mixing Phase C components, namely, Creammaker Blend, Ceteareth-25, Glyeral Stearate, Olive Oil Pure, and Almond Oil, and heating the mixture to 75-80 degrees Celsius. At block 310, the method 300 includes adding the Phase A/B mixture to the Phase C mixture with homogenizing/high shear mixing. At block 312, the method 300 includes adding Phase D components, namely, the Moroccan and kaolin clay, among other supporting powder ingredients: Cassia Obovata, Hibiscus Powder, Neem Powder, Bacopa Powder, Gotu Kola Powder Organic, Fenugreek Seed Powder, Amia Powder, and Moringa Leaf Powder, one at a time to the A/B/C mixture.

At block 314, the method 300 includes cooling the combined mixture to 45 degrees Celsius. At block 316, the method 300 includes adding Phase E components, namely, Vitamin E, Sweet Orange Essential Oil, Lemongrass Oil, Bhringraj Oil, Lavendar Oil, FSS Green Tea Extract, FSS White Willow Bark Extract, DL-Panthenol, Aloe Vera Powder, and Organic Aloe Vera Gel to the A/B/C/D mixture. At block 318, the method 300 includes ensuring the pH is in a range of 3.5 to 3.8.

It will be appreciated that various embodiments of the above-disclosed and other features and functions, or alternatives or varieties thereof, may be desirably combined into many other different systems or applications. Also, that various presently unforeseen or unanticipated alternatives, modifications, variations or improvements therein may be subsequently made by those skilled in the art which are also intended to be encompassed by the following claims.

1. A hair cleanser formulation for textured hair, comprising:
from 15% to 25% by weight of one or more clays; and
from 3% to 7% by weight of glycolic acid, wherein the glycolic acid combines with the clays from 18% to 32% by weight of the of the formulation, and wherein supporting ingredients make up the remaining total volume (% w/w) of the formulation.

2. The hair cleanser formulation of claim 1, wherein the clays include a first clay selected from a smectite clay group and a second clay selected from a montmorillonite clay group.

3. The hair cleanser formulation of claim 2, wherein the first clay is kaolin clay and the second clay is Moroccan clay.

4. The hair cleanser formulation of claim 3, wherein the hair cleanser formulation includes 1%±0.01 weight percent of the kaolin clay.

5. The hair cleanser formulation of claim 3, wherein the hair cleanser formulation includes 20%±0.01 weight percent of the Moroccan clay.

6. The hair cleanser formulation of claim 1, wherein the hair cleanser formulation has 3% ±0.01 glycolic acid.

7. The hair cleanser formulation of claim 1, wherein the hair cleanser formulation has a pH from 3.5 to 3.8.

8. The hair cleanser formulation of claim 1, wherein the hair cleanser formulation has a pH of 3.8±0.01.

9. The hair cleanser formulation of claim 1, wherein the supporting ingredients are selected from the group consisting of: sodium phytate, sodium benzoate, potassium hydroxide, xanthan gum, glyceryl stearate, PEG-100 stearate, olea Europaea (olive) oil, Prunus amygdalus dulus (Sweet Almond) oil, cassia italica leaf powder, hibiscus sabdariffa, antelea azadriratcha indica (neem) powder, and panthenol.
